(19) United States
(12) Patent Application Publication
(10) Pub. No.: US 2007/0060846 A1
(43) Pub. Date: Mar. 15, 2007

(54) Multiple stage wire guide

(75) Inventor: David M. Hardin, Winston-Salem, NC (US)

Correspondence Address: BRINKS HOFER GILSON & LIONE/CHICAGO/COOK, PO BOX 10395, CHICAGO, IL, 60610, US

(73) Assignee: Wilson-Cook Medical Inc., Winston-Salem, NC (US)

(21) Appl. No.: 11/519,253

(22) Filed: Sep. 12, 2006

Related U.S. Application Data
(60) Provisional application No. 60/718,097, filed on Sep. 15, 2005.

(51) Int. Cl. A61M 25/00

(52) U.S. Cl. 600/585

(57) ABSTRACT

A wire guide device includes an elongate wire guide having a proximal end and a distal end and a coating affixed to a portion of the wire guide. The coating includes at least two different diameters alternating between the proximal end and the distal end of the wire guide along the length of the wire guide. A wire guide device further includes an elongate wire guide comprising a first portion having a first diameter, a second portion having a second diameter, and a third portion having a third diameter alternating between a proximal end and a distal end of the wire guide along at least a portion of the wire guide.

### RELATED APPLICATIONS

This application claims priority to provisional application No. 60/718,097 filed on Sep. 15, 2005, the entire disclosure of which is incorporated by reference herein.

### FIELD OF THE INVENTION

The present invention relates to the field of medical devices, and more particularly to medical devices such as wire guides used during medical procedures to define a path within the body of a patient and allow surgeons to use that path for access with diagnostic or therapeutic instruments.

### BACKGROUND

Wire guides are used during many medical procedures in the gastrointestinal system, including the pancreatobiliary system (i.e., the biliary tree), the stomach, and the esophagus. During vascular procedures, such as balloon angioplasty, stent placement, and endoluminal grafts for aortic aneurysms, the use of wire guides are essential in assessing the site of the particular obstruction in the affected artery. Wire guides are long, slender, relatively flexible wires that are used to gain and maintain access to the body's narrow passageways during minimally invasive medical procedures. Because of the substantial length and width of the wire guide, it can be cumbersome and require constant, delicate manipulation by a physician.

Due to the complexity of many types of medical procedures, physicians often need to maneuver other medical instruments over the wire guide during different stages of the particular medical procedure. Typically, surgeons wishing to introduce a catheter or other diagnostic or therapeutic instrument into such a passageway or vessel utilize the Seldinger technique which encompasses the exchange of instruments over a wire guide. One accepted method is to first introduce a wire guide into the patient, working the wire guide into the body of the patient in a minimally-invasive manner. The wire guide is then used to guide other medical instruments into the patient.

Navigating the wire guide through the body vessel(s) of the patient may be difficult. Often, the body vessel defines a torturous path due to the presence of natural bends or curves in the body vessel, or unnatural impediments such as tumors, build-ups, and/or strictures may also be present. The presence of a torturous path may make navigation of a wire guide difficult. For example, the presence of an impediment may block the wire guide from navigating further into the vessel to reach the repair site.

The friction experienced when advancing a wire guide within a lumen of a medical device, or when advancing a medical device over the wire guide, is dependent on the size, or diameter, of the wire guide relative to the lumen. That is the amount of friction depends on how much surface area of the wire guide makes physical contact with the lumen. Wire guides are available in a variety of sizes, shapes, and diameters, which result in varying levels of friction with the lumen during advancement. Minimal friction is desired to decrease the incidence of damage to the lumen as well as to facilitate the ease with which the wire guide is advanced and utilized.

In addition, wire guides often must be maintained in a stationary position relative to the patient while a physician performs various procedures. In particular, maintaining the wire guide in a stationary position is important to prevent loss of access to a target anatomy, for example, a duct in the biliary tree. Also, during an esophageal dilation, a physician must secure a wire guide within the esophagus and across an esophageal stricture as one or more dilators are advanced over the wire guide. Likewise, during a percutaneous endoscopic gastromy (PEG) tube placement, a wire guide must be secured relative to the patient's mouth, esophagus, and stomach as a physician inserts a feeding tube. However, maintaining the wire guide stationary may be difficult, particularly, when manipulating other medical instruments over the wire guide, as a result of friction between the wire guide and the other instruments.

What is needed is a wire guide device that can be quickly and easily advanced through a lumen, requires minimal effort to advance through the lumen during usage, reduces friction during positioning and maneuvering of the wire guide through the lumen, and can be maintained in a stationary position while manipulating other medical instruments over the wire guide. Also, the wire guide device should require minimal time and effort to operate during medical procedures.

### SUMMARY

In one aspect of the invention, a wire guide device is provided that comprises an elongate wire guide having a proximal end and a distal end and a coating affixed to a portion of the wire guide. The coating includes at least a first thickness and a second thickness alternating between the proximal end and the distal end of the wire guide along at least a portion of the wire guide. The coating can be applied on a straight scale or on a diagonal scale. Applying the coating on a diagonal scale provides increased flushing ability after the wire guide has been inserted through a lumen.

The elongated wire guide provides a first portion having a first diameter, and the alternating coating provides a second portion having a second diameter and a third portion having a third diameter. The alternating second and third diameters provided by the coating and the first diameter provided by the elongated wire guide combine to provide a multiple stage wire guide wherein the first diameter is smaller than the second diameter, and the third diameter is larger than the second diameter.

The diameter provided by the third portion of the coating is configured to engage a lumen so as to reduce the friction along the lumen. The coating of the wire guide device can include shapes selected from the group consisting of circular, round, oval, and square, triangular, rectangular, pentagonal and hexagonal. The coating of wire guide device can further provide a textured surface for engaging a lumen. Additionally, the alternating diameter of the wire guide reduces the contact surface area of the wire guide device thereby increasing the overall maneuverability of the device. In other configurations, the wire guide can provide diameters providing wire guides having two, four and more stages.

In yet another aspect of the invention, a wire guide device is provided that comprises an elongate wire guide having a proximal end and a distal end, wherein the elongated wire guide comprises a first portion having a first diameter, a second portion having a second diameter, and a third portion having a third diameter alternating between the proximal end and the distal end of the wire guide along at least a portion of the wire guide. In this embodiment, the elongated wire guide provides a triple stage effect by including alternating diameters in the core of the wire guide wherein the first diameter is smaller than the second diameter and the third diameter is larger than the first diameter and second diameter. The third portion is configured to engage a lumen so as to reduce the friction along the lumen. The shapes of the portions of the wire guide can be selected from the group consisting of circular, round, oval, and square, triangular, rectangular, pentagonal and hexagonal.

In another aspect of the invention, a wire guide device is provided that comprises a first plurality of diameters, a second plurality of diameters, and a third plurality of diameters, wherein the first, second and third plurality of diameters are arranged in an alternating fashion along a portion of wire guide.

### BRIEF DESCRIPTION OF THE DRAWINGS

Several embodiments of the present invention will now be described by way of example with reference to the accompanying drawings, in which:

FIGS. 1A-1F show a perspective view of a wire guide device according to an embodiment of the present invention;

FIG. 2 shows a cross-sectional view of the wire guide device of FIG. 1A;

FIG. 3 shows a perspective view of the wire guide of FIG. 1A inserted into a sheath;

FIG. 4 shows a perspective view of a wire guide device according to an embodiment of the present invention;

FIG. 5 shows a sectional view of the wire guide of FIG. 4 inserted into a lumen;

FIG. 6 shows a perspective view of a wire guide device according to an embodiment of the present invention;

FIG. 7 shows a cross-sectional view of the wire guide device of FIG. 6;

FIG. 8 shows a perspective view of a wire guide device according to an embodiment of the present invention;

FIG. 9 shows a cross-sectional view of the wire guide device of FIG. 8; and

FIG. 10 shows a perspective of a wire guide device according to an embodiment of the present invention.

### DESCRIPTION OF THE INVENTION

The invention is described with reference to the drawings in which like elements are referred to by like numerals. The relationship and functioning of the various elements of this invention are better understood by the following detailed description. However, the embodiments of this invention are not limited to the embodiments illustrated in the drawings. It should be understood that the drawings are not to scale, and in certain instances details have been omitted which are not necessary for an understanding of the present invention, such as conventional fabrication and assembly.

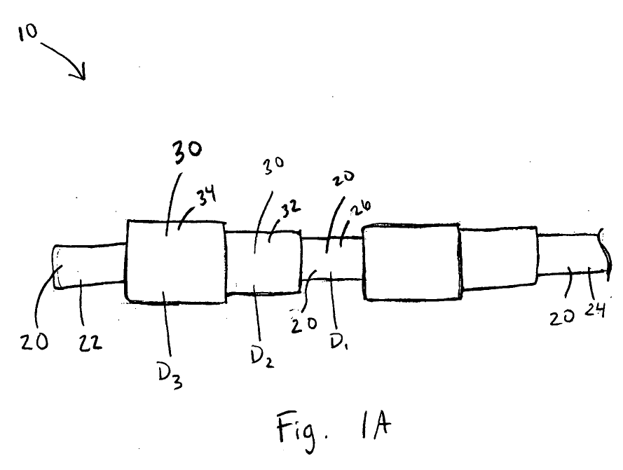

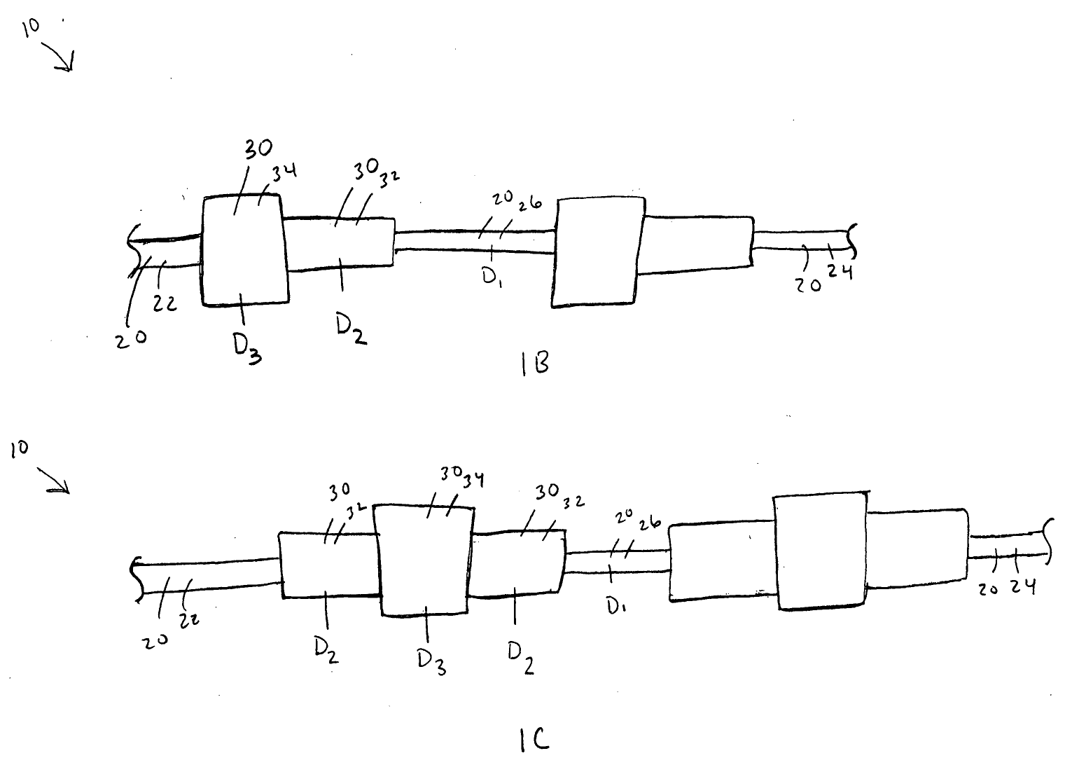

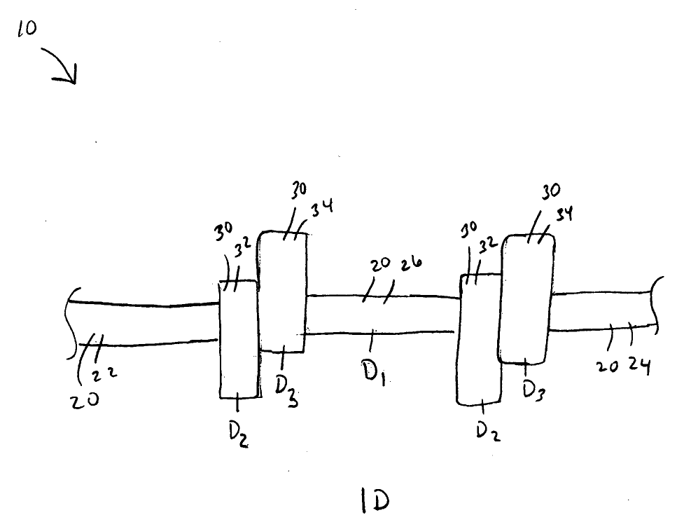

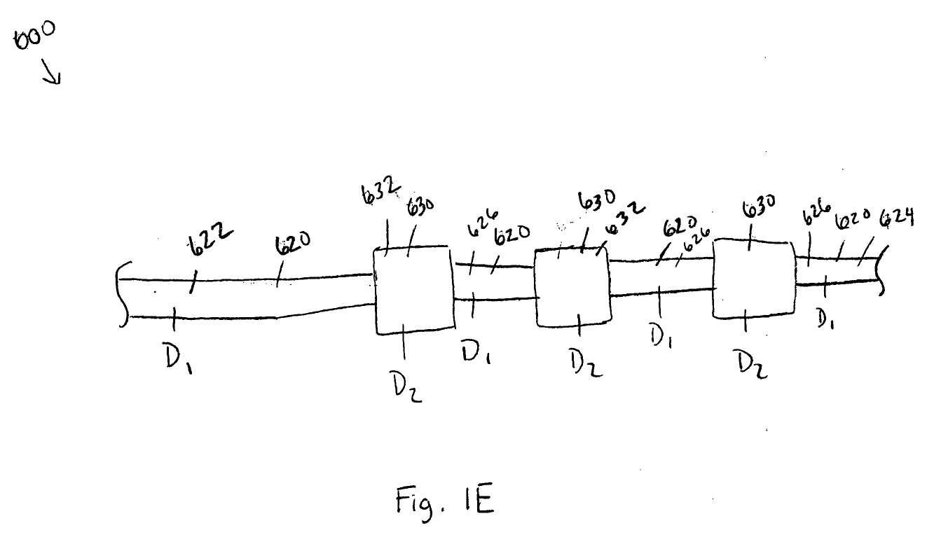

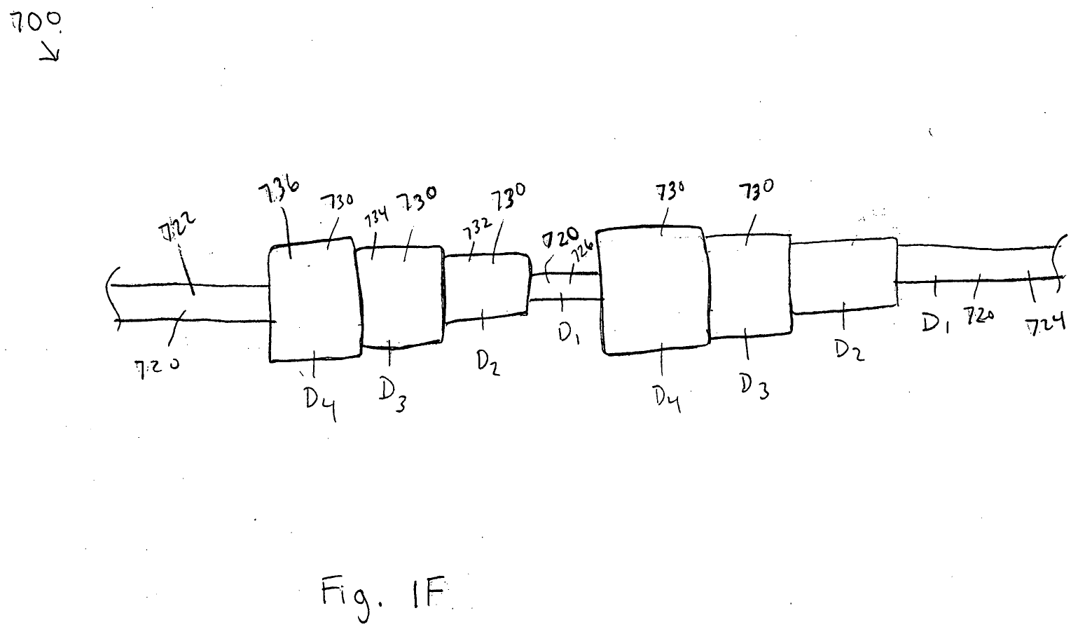

The wire guide device of the present invention depicted in FIGS. 1-10 comprises one or more wire guides, each comprising varying diameters alternating between a proximal end and a distal end of the wire guide along at least a portion of the wire guide. The alternating diameters of the wire guide can be comprised of a coating and/or a core section of the wire guide. The coating of the wire guide can be applied utilizing a straight scale or a diagonal scale.

As used in the specification, the terms proximal and distal should be understood as being in the terms of a physician using the wire guide. Hence the term distal means the portion of the wire guide which is farthest from the physician and the term proximal means the portion of the wire guide which is nearest to the physician.

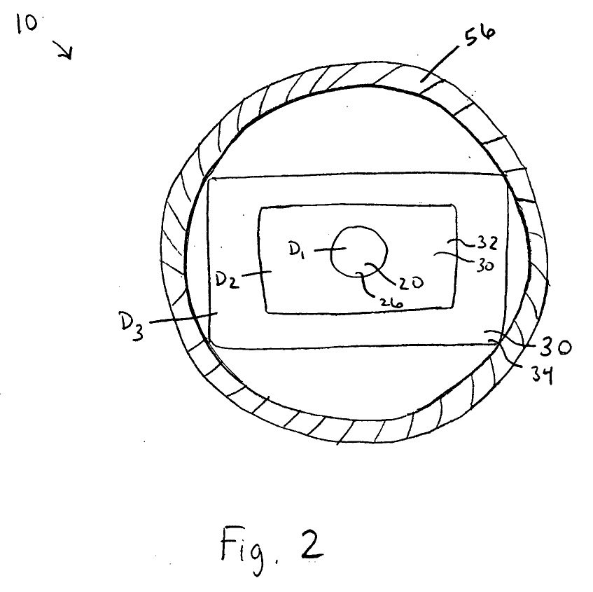

Referring now to FIGS. 1A-1D, an embodiment of a wire guide device 10 of the present invention is shown. The wire guide device 10 includes an elongated wire guide 20 having a proximal end 22 and a distal end 24 and a coating 30 affixed to a portion of the wire guide 20. The coating 30 has been applied using a straight scale. The wire guide 20 further includes a first portion 26, wherein the first portion 26 includes a first diameter D1. The coating 30 comprises a second portion 32 and a third portion 34, wherein the second portion 32 includes a second diameter D2 and the third portion 36 includes a third diameter D3. The alternating diameters of the device 10 provide a multiple stage wire guide 20 wherein the first diameter D1 is smaller than the second diameter D2. Additionally, the third diameter D3 is larger than the first diameter D1 and the second diameter D2. The third portion 34 of the wire guide device 10 is configured to engage a lumen of a medical device (see FIG. 2) such as a catheter, so as to reduce the friction along the lumen. Also, the thicknesses of the coating 30 alternates along the varying portions of the wire guide 20. The multiple stage wire guide 20 provided by the alternating diameters facilitates the passage of fluid over the wire guide 20 and increases maneuverability of the wire guide 20 after being inserted through the lumen.

Referring to FIGS. 1E-F, yet another embodiment of a wire guide device 600, 700 of the present invention is shown. Particularly, FIG. 1E depicts a wire guide device 600 having a double stage wire guide 620 having a proximal end 622 and a distal end 624 and a coating 630 affixed to a portion of the wire guide 620. The coating 630 has been applied using a straight scale. The wire guide further comprises a first portion 626, wherein the first portion 626 includes a first diameter D1 and a second portion 632, wherein the second portion 632 includes a second diameter D2. The alternating diameters of the device 600 provide a double stage wire guide 20 wherein the first diameter D1 is smaller than the second diameter D2. FIG. 1F depicts a wire guide device 700 having a quadruple stage wire guide 720 comprising a proximal end 722 and a distal end 724 and a coating 730 affixed to a portion of the wire guide 720. The coating 730 has been applied using a straight scale. The wire guide device 700 comprises a first portion 726, wherein the first portion 726 includes a first diameter D1 and a second portion 732, wherein the second portion 732 includes a second diameter D2. Additionally, the wire guide device 700 comprises a third portion 734, wherein the third portion 734 includes a third diameter D3 and a fourth portion 736, wherein the fourth portion 736 includes a fourth diameter D4. The alternating diameters of the device 700 provide a quadruple stage wire guide 720 wherein the varying diameters are configured to engage a lumen of a medical device so as to reduce the friction along the lumen. Alternate embodiments of the present invention can include configurations and designs providing a multiple stage wire guide of varying stages. For example, a multiple stage wire guide can comprise five, six or more stages depending on the size and configuration of the device. Also, the multiple stages of the wire guide can include diameters and portions that provide color coding to aid in the visualization of the wire guide during medical procedures such as endoscopy procedures. For instance, a triple stage wire guide can include a first stage, second stage and third stage wherein the first stage is red, the second stage is blue and the third stage is white to provide a visual aid to a physician when advancing the wire guide to a lumen of the patient. One of ordinary skill in the art would appreciate that the device can include other designs and color combinations.

FIG. 2 illustrates a cross-section of the wire guide device 10 of FIG. 1A having alternating diameters along at least a portion of the wire guide 20. In the embodiment illustrated, the shape of the first portion 26 is circular, the second portion 32 is rectangular, and the third portion 34 is rectangular. Additionally, the third diameter D3 provides the largest diameter of the wire guide device 10. The shape and size of the third portion 34 minimizes contact with the lumen 56 of the elongate medical device (e.g. a catheter), as well as providing a passageway through which fluid may pass around the wire guide 20. The alternating diameters of the wire guide device 10 also allows the wire guide device 10 to be freely inserted and removed from the lumen 56 during medical procedures. Upon insertion of the wire guide device 10 into the lumen 56, the third portion 34 of the wire guide device 10 minimizes lateral movement of the wire guide 20 when positioned therein while also allowing the elongate wire guide 20 to be maneuvered longitudinally along the lumen 56 for purposes of introduction to a target site of the patient. The level of friction provided by engaging only a portion of the wire guide 10 to the lumen 56 should not be so great as to prevent or inhibit relatively easy insertion or withdrawal of the wire guide 20 from the lumen 56.

The varying thickness of the coating 30 further enables a physician to easily grasp and operate the device 10 during positioning and maneuvering of the wire guide 20 due to the varying surface area along the device 10. The varying thickness can provide a tactile feel to the physician to withdraw, turn or hold the device 10 in a stationary position. The varying thickness of the coating 30 combined with the varying diameter of the wire guide 20 also provides a visual indication as the device 10 is viewed through an endoscope during maneuvering. It should be appreciated that other designs utilizing different diameters could also be utilized. The wire guide device 10 is likewise not limited to one particular shape, but can comprise varying shapes depending on the particular use. The shapes of the constituent components can be selected from the group consisting of circular, round, oval, square, triangular, rectangular, pentagonal, hexagonal or any other suitable shape.

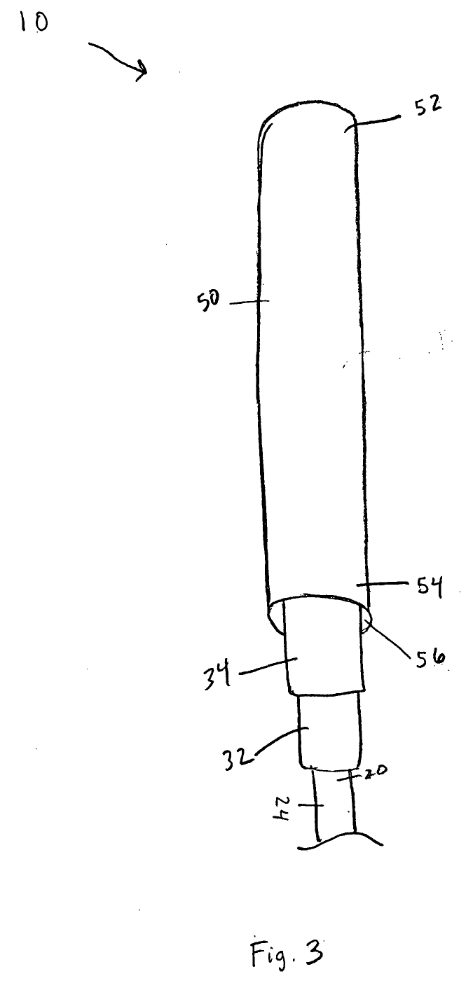

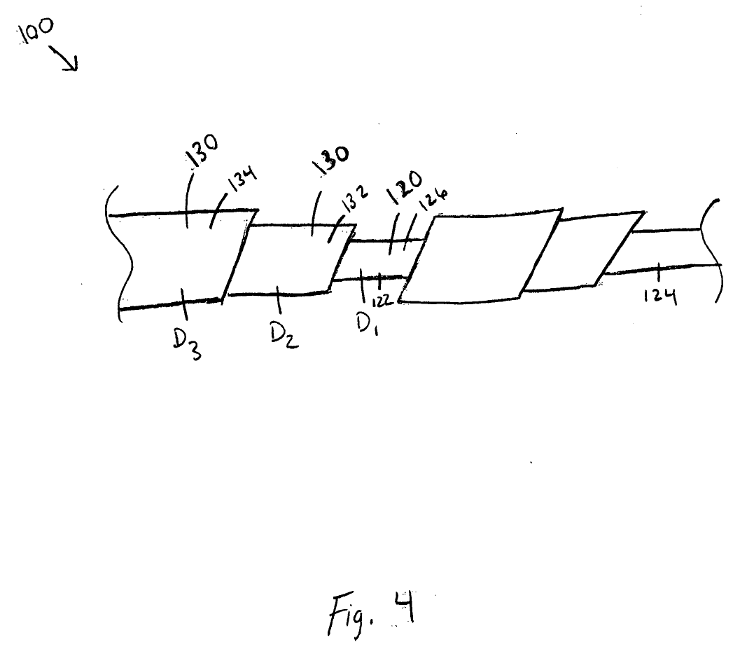

FIG. 3 illustrates an elongate medical device, e.g. a catheter or introducer sheath 50 enclosing the wire guide device 10 of FIG. 1A. The introducer sheath 50 comprises a proximal end 52, a distal end 54, and a lumen 56 extending through at least a portion thereof. The introducer sheath 50 is configured to receive and hold the wire guide 20, or similar type of elongate medical device. The wire guide 20 is inserted through the lumen 56 of the introducer sheath 50 from either the distal end 54 or the proximal end 52. The introducer sheath 50 may be formed from a polyethylene material and may have a lubricous coating. Different materials and surface treatments may also be considered for introducer sheath 50.

The largest diameter of the wire guide 20 is preferably the only portion that engages the lumen 56 of the introducer sheath 50. The total contact surface area of the wire guide device 10 is thereby substantially reduced as only the third portion 34 engages the lumen 56. The total contact surface area of the wire guide device 10 is also dependent on the respective lengths of the first portion 26, second portion 32 and third portion 34 as the relative lengths of the portions can vary (see FIG. 1B). Additionally, the positioning of the respective portions may vary as illustrated in FIG. 1C, wherein the second portion 32 is positioned on both sides of the third portion 34. In another embodiment illustrated in FIG. 1D, the second portion 32 and third portion 34 may be positioned in an offset configuration along a portion of the wire guide 20. In a preferred embodiment, the diameter of the wire guide 20 may range between about 0.005″ and 0.040″. Preferably, the largest diameter of the wire guide 20 is 0.035″. In alternate embodiments the diameter of the wire guide 20 may vary depending on the design and intended use.

The wire guide 20 may be preferably formed of molded plastic material. Alternatively, the wire guide 20 and other component parts of the device 10, such as the coating 30 may be formed of any other material, including metal, as desired by an individual. It should be noted that the component parts of the wire guide device 10 may be formed of a material which may be repeatedly sterilized by medical providers for re-use during medical procedures. Alternatively, the wire guide device 10 may be sterilized for initial use only, and disposed of following use as desired by an individual.

Variations of the types of coatings applied to the wire guide may include polymers or metals. Specifically, a polymer coating can be disposed along the wire guide in a straight scale (see FIG. 1A) or diagonal scale (see FIG. 4). The coating 30 can further be treated by a mechanical abrasion process or chemical etching by an acid or other chemical which results in texturing of the outer surface of the coating 30. The coating 30 thereby creates a mechanical interlock between the surface of the wire guide 20.

Varying amounts of the coating 30 can be applied to the wire guide 20 depending on the specific configuration and shape of the embodiment. The quantities of coating 30 may be discreet and unconnected to one another to thereby form a non-continuous coating 30 comprising a system of shapes with varying thicknesses. The coating 30 may be deposited onto the wire guide 20 so that the coating 30 extends continuously along a portion of the wire guide 20. In one embodiment, a Teflon coating may be applied to the wire guide 20. Alternatively, the coating 30 may be a swellable or expandable coating such as a hydrophilic material (e.g., a hydrogel). This type of coating 30 may be initially deposited onto the wire guide 20 such that the surface of the coating 30 provides uniform thickness as the diameter alternates from a smaller to larger size. The coating 30 may be a hybrid polymer mixture, such as those based on polyvinyl puroladine and cellulose esters in organic solvent solutions. These solutions make the wire guide device 10 particularly lubricous when in contact with body fluids, which aids in navigation.

The coating 30 can include a textured surface for engaging a lumen. The textured surface provides additional support at the point of engagement between the wire guide device 10 and the lumen. The coating 30 of the wire guide 20 may further provide the wire guide 20 with increased strength or flexibility, or may assist in reducing any associated friction along the length of the wire guide 20 during a medical procedure.

Referring to FIG. 4, an embodiment of a wire guide device 100 is illustrated wherein a coating 130 is applied having a diagonal scale. The coating 130 includes a second portion 132 and a third portion 134 having a rectangular shape. The second portion 132 includes a second diameter D2 and the third portion 134 includes a third diameter D3. The wire guide 120 includes a first portion 126 having a first diameter D1 wherein the first diameter D1 is smaller than the second diameter D2. Additionally, the third diameter D3 is larger than the first diameter D1 and the second diameter D2. The different diameters alternate between a proximal end 122 and a distal end 124 of the wire guide 120 with each portion having a diagonal scale. The wire guide 120 may include several coatings that alternate along at least a portion of the wire guide 120. In addition, the thicknesses of the coatings 130 alternate along the varying portions of the wire guide 120. The optimal length of the wire guide 120 is determined by considering factors such as design and material used, as well by what is determined through experimentation to work best.

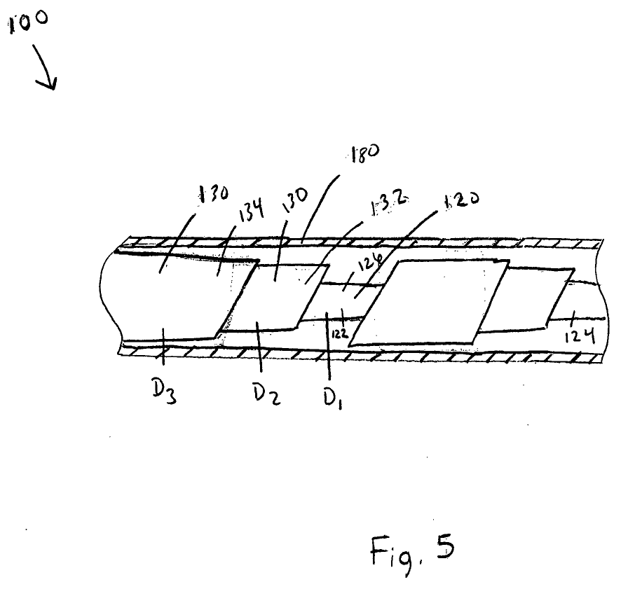

FIG. 5 illustrates a view of the wire guide device 100 of FIG. 4 inserted through a lumen 180 of an elongate medical device. The diagonal scale of the coating 130 and the wire guide 120 provides an increase in the flushing ability of the wire guide device 100. The diagonal scale of the alternating diameters also produces a spiraling effect along the body of the wire guide device 100 which allows fluid to easily pass over the triple stage diameter of the device 100 during flushing. The alternating triple stage diameter of the device 100 has a larger unoccupied volume than a double stage device which allows fluid to flow at an increased rate during flushing. Additionally, flushing fluid has more space for movement as the device 100 provides a decrease in contact surface area as only a portion of the device 100 is engaged to the lumen 180.

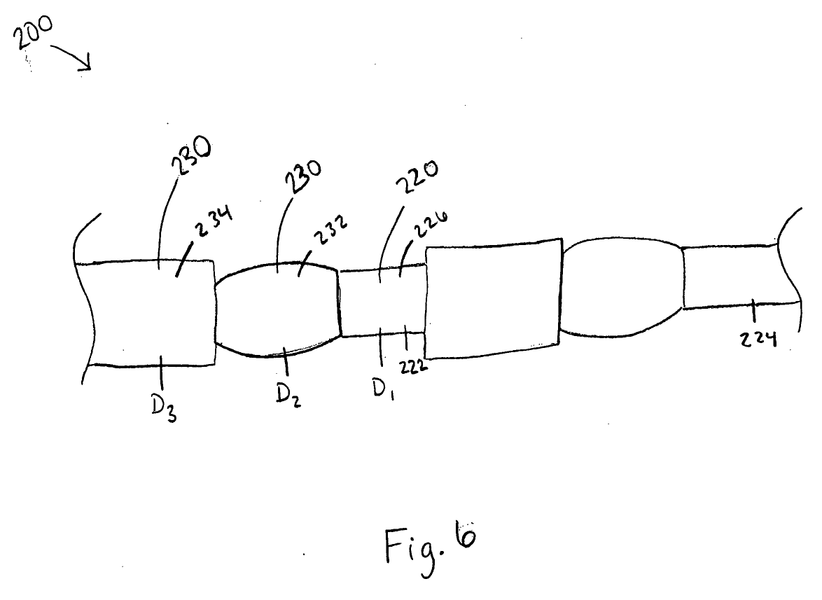

Referring to FIG. 6, an embodiment of the wire guide device 200 is illustrated wherein a wire guide 220 comprises a first portion 226 and a coating 230 including a second portion 232 and third portion 234. The coating 230 is applied using a straight scale. In the embodiment illustrated the first portion 226 provides a first diameter D1 having a circular shape, the second portion 232 provides a second diameter D2 having an oval shape, and the third portion 234 provides a third diameter D3 having a rectangular shape. The third diameter D3 of the rectangular third portion 234 may be larger than the diameters D2, D1 provided by the oval second portion 232 and the circular first portion 226.

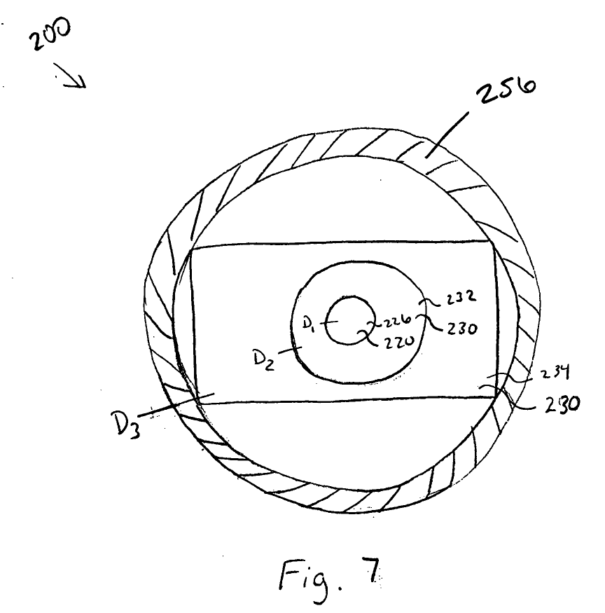

FIG. 7 illustrates a cross-section of the wire guide device 200 of FIG. 6 inserted through a lumen 256 of an elongate medical device; or body vessel. Normally when a wire guide 220 is inserted through the lumen 256 the entire wire guide 220 is fully engaged to the lumen 256 causing an increase in the level of friction between the lumen 256 and the wire guide 220. The portion of the wire guide 220 engaged to the lumen 256 provides friction by pressing against the lumen 256 to prevent the flexible wire guide 220 from inadvertently slipping once it is positioned. The varying triple stage diameter provided by the coating 230 allows only a portion of the wire guide 220 to engage the lumen 256 of a medical device or body vessel. The reduced level of friction in the lumen 256 allows the wire guide 220 to be maneuvered and positioned freely along the lumen 256. In particular, the third portion 234 of the wire guide device 200 is engaged to the lumen 256 to minimize lateral movement of the wire guide 220 when positioned therein while also allowing the elongate wire guide 220 to be maneuvered longitudinally along the lumen 256 for purposes of introduction to a target site of the patient. The level of friction provided by engaging only a portion of the wire guide 220 to the lumen 256 should not be so great as to prevent or inhibit relatively easy insertion or withdrawal of the wire guide 220 from the lumen 256.

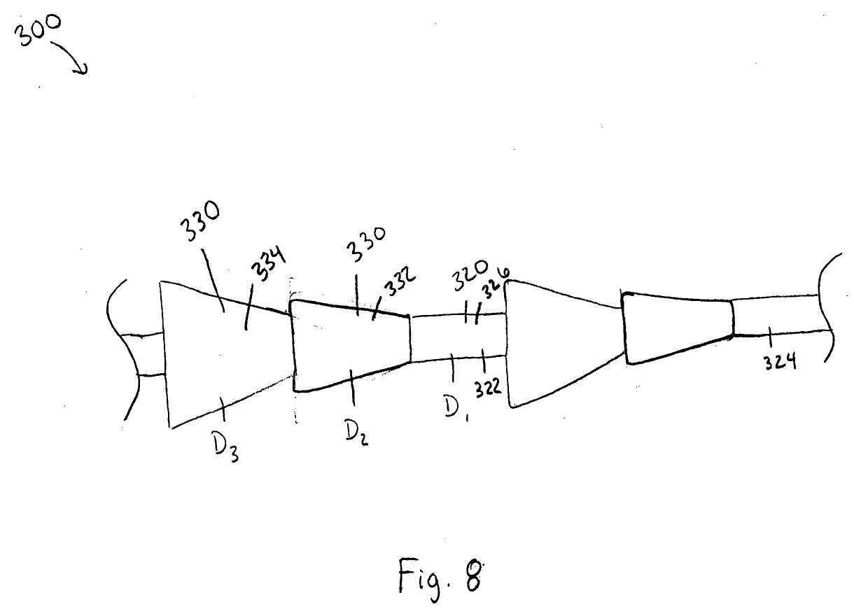

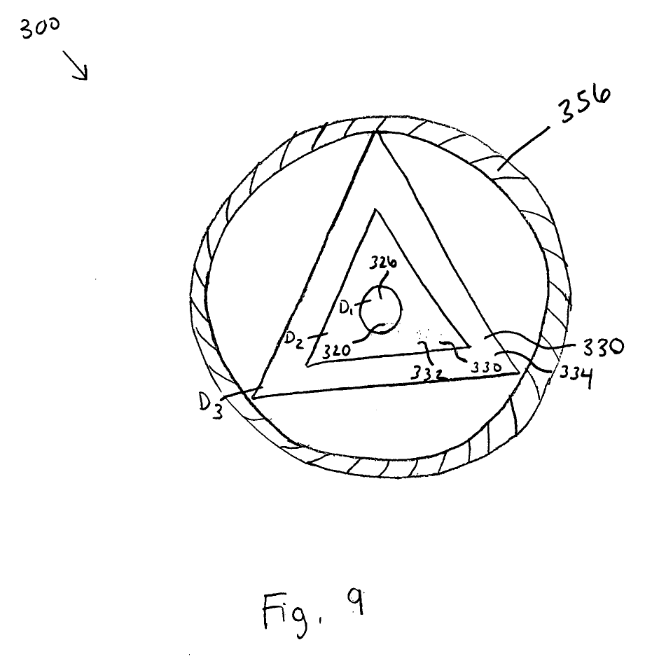

FIG. 8 illustrates an embodiment of a wire guide device 300 wherein a wire guide 320 comprises a first portion 326 and a coating 330, wherein the coating 330 further comprises a second portion 332 and third portion 334 having a triangular shape. The coating 330 is applied using a straight scale. FIG. 9 illustrates a cross-section of the wire guide device 300 of FIG. 8. The outermost diameter D3 of the triangular third portion 334 is larger than the outermost diameter D2 of the triangular second portion 332, both of which are larger than diameter D1 of the circular first portion 326. The triangular shape of the third portion 334 of the coating 330 is sized and configured to align with a lumen 356 so as to allow only the edges of the third portion 334 to engage the lumen 356. In particular, the third portion 334 of the wire guide device 300 is engaged to the lumen 356 to minimize lateral movement of the wire guide 320 when positioned therein while also allowing the elongate wire guide 320 to be maneuvered longitudinally along the lumen 356 for purposes of introduction to a target site of the patient. The triangular configuration significantly reduces the level of friction between the surface of the wire guide device 300 and the lumen 356.

In the illustrative embodiment shown in FIG. 8, the triangular coating 330 can comprise polytetrafluoroethylene, or another suitable material. Examples of suitable coverings include fluoropolymers, polyurethanes, and other suitable coatings used in the medical device arts. The coating 330 may be applied by dipping, molding, or spraying a suitable coating material, such as polytetrafluoroethylene, urethane, and/or other polymeric coatings directly to the desired portions of the wire guide device 300. Alternatively, the coating 330 may be applied by heat shrinking a heat shrinkable material about the desired portions of the wire guide device 300.

One preferred coating 330 comprises a thin PTFE heat shrinkable material. The heat shrinkable nature of these materials facilitates manufacturing while providing a lubricious coating, which facilitates navigation. In a preferred embodiment, the coating 330 may be applied to a wire guide device 300 having a preformed tube configuration that is heat shrinkable or otherwise bonded to the device 300. The coating 330 may be applied onto the wire guide device 300 as a sheet, or coated in sections onto the wire mandrel or a mold. For example, the wire guide device 300 can include a coating having a first thickness applied by heat shrinking and a second a second thickness applied by bonding, wherein the thicknesses of the coating alternates between a proximal end and a distal end of a wire guide along at least a portion of the wire guide. The thicknesses of the coating can vary according to the particular design and desired specifications of the device 300. A skilled artisan would appreciate that other embodiments could include varying designs comprising different shapes and thicknesses, along with different coatings. Of course, the coating 330 could be eliminated entirely.

The wire guide device 300 may be manufactured by selectively removing material in discrete bands from the coating 330 of the wire guide device 300 to form different diameter portions of the wire guide device 300. The different diameters of the wire guide device 300 may be formed by shaving, buffing, grinding, etching, or otherwise removing material from the single coating 330. For example, a wire guide device 300 may comprise a wire guide initially comprising only a first diameter that is chemically etched to thereby form a device 300 comprising a second diameter and third diameter. Alternatively, other embodiments may comprise a core wire guide that is grounded, etched, or otherwise shaped to form the multiple diameter portions of the wire guide device 300. In this embodiment, the coating 330 may be applied to the wire guide device 300 by dipping or spraying.

Radiopaque materials known in the art including, but not limited to, bismuth or gold can be added in the coating of any embodiments of the present invention. Also, radiopaque markers known in the art can be affixed to the device 300. Several examples of suitable radiopaque materials and markers are known in the art, and any suitable material and/or marker can be utilized in the present invention.

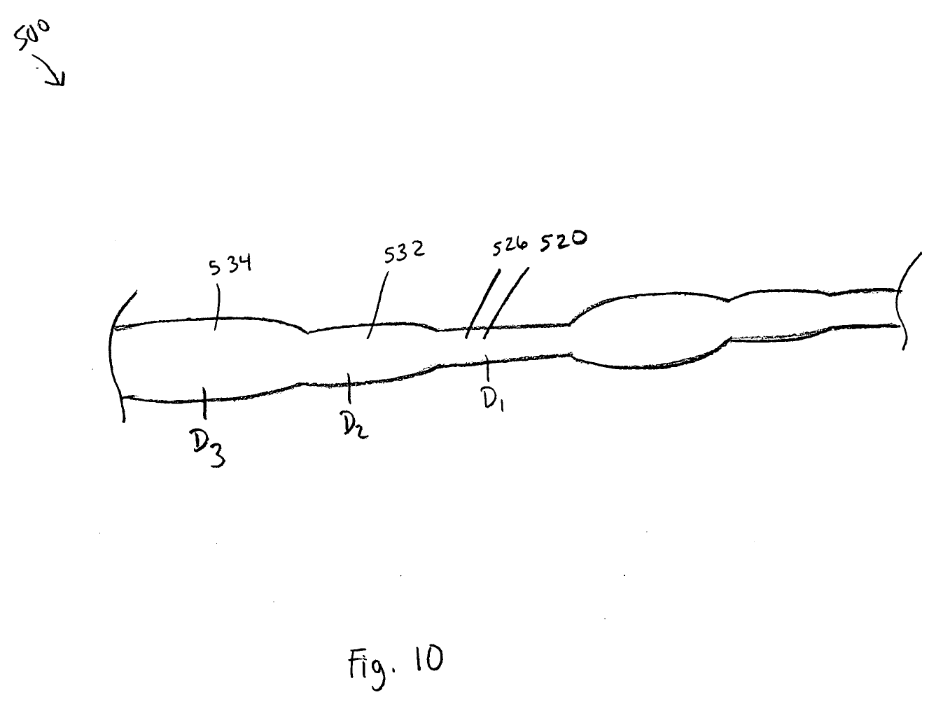

FIG. 10 depicts yet another embodiment of a wire guide device 500 of the present invention. In this embodiment, the elongate wire guide 520 comprises a proximal end and a distal end, wherein the core wire guide 520 further comprises a first portion 526 having a first diameter D1, a second portion 532 having a second diameter D2 and a third portion 534 having a third diameter D3 alternating between the proximal end 522 and the distal end 524 of the wire guide 520 along the length of the wire guide 520. The alternating diameter creates a triple stage comprised entirely of the elongated wire guide 520 instead of a coating being applied to create the alternating diameters.

The wire guide 520 having a core comprising alternating diameters provides many advantages during the delivery of medical devices to an obstructed destination site within a patient's body cavity. The core of the wire-guide 520 provides increased flexibility to the device 500 along with providing increased flushing ability when utilized in a lumen. The multiple stage wire guide device 500 can be created by machining, crimping or deforming. The multiple stage wire guide device 500 also maintains the ability to effectively manipulate the transport of any available alternative medical device through the lumen or body cavity of a patient.

Any suitable material can be used for the core structure of the wire guide 520 and a variety of suitable materials are known to those skilled in the art. The material chosen need only be biocompatible and able to be formed into the structures described herein. Examples of suitable materials include stainless steel, nitinol and other nickel-titanium alloys, MP35N® and other nickel-cobalt alloys, Cobalt L-605™ and other cobalt-chromium alloys, other biocompatible metals, metal-alloys, as well as polymeric materials. The interior surface of the wire guide 520 can be a solid wire or made from a material similarly suitable for acute use in the human body. The core of the wire guide 520 can be made of the same material uniformly or from multiple materials having different inherent property characteristics.

Preferably, the wire guide device 520 can comprise a tubular member or a sheet of material. The device 520 can also be formed from a series of layers, or as a coated core structure. For example, the wire guide 520 can comprise nitinol with a solid core in one embodiment or a nitinol core with a polytetrafluoroethylene covering in another embodiment. Depending on the desired range of movement of the wire guide 520, the appropriate material can be selected and configured as needed.

Any other undisclosed or incidental details of the construction or composition of the various elements of the disclosed embodiment of the present invention are not believed to be critical to the achievement of the advantages of the present invention, so long as the elements possess the attributes needed for them to perform as disclosed. The selection of these and other details of construction are believed to be well within the ability of one of even rudimentary skills in this area, in view of the present disclosure. Illustrative embodiments of the present invention have been described in considerable detail for the purpose of disclosing a practical, operative structure whereby the invention may be practiced advantageously. The designs described herein are intended to be exemplary only. The novel characteristics of the invention may be incorporated in other structural forms without departing from the spirit and scope of the invention.

1. A wire guide device, comprising:
an elongate wire guide having a proximal end and a distal end; and
a coating affixed to a portion of the wire guide, wherein the coating includes at least a first thickness and a second thickness alternating between the proximal end and the distal end of the wire guide along at least a portion of the wire guide.

2. The wire guide device of claim 1 wherein the coating is applied on a straight scale.

3. The wire guide device of claim 1 wherein the coating is applied on a diagonal scale.

4. The wire guide device of claim 1 wherein the coating includes a cross-sectional shape selected from the group consisting of circular, round, oval, and square.

5. The wire guide device of claim 1 wherein the coating includes a cross-sectional shape selected from the group consisting of triangular, rectangular, pentagonal and hexagonal.

6. The wire guide device of claim 1, wherein the wire guide device includes a first portion having a first diameter, a second portion having a second diameter and a third portion having a third diameter.

7. The wire guide device of claim 6 wherein the first diameter is smaller than the second diameter.

8. The wire guide device of claim 6 wherein the third diameter is larger than the first diameter and the second diameter.

9. The wire guide device of claim 6, wherein the third portion is configured to engage a lumen so as to reduce friction between the wire guide device and the lumen.

10. The wire guide device of claim 1 wherein the coating includes a textured surface for engaging a lumen.

11. The wire guide device of claim 1 wherein the wire guide comprises a material selected from the group consisting of nitinol, stainless steel and titanium.

12. The wire guide device of claim 1 wherein the coating comprises a material selected from the group consisting of polyvinyl puroladine, cellulose, polytetraflouroethylene and urethane.

13. The wire guide device of claim 1 wherein the alternating diameter of the wire guide reduces the contact surface area of the wire guide device.

14. The wire guide device of claim 1 wherein the wire guide has a diameter ranging between 0.005″ and 0.040″.

15. A wire guide device, comprising:
an elongate wire guide having a proximal end and a distal end, wherein the wire guide comprises a first portion having a first diameter, a second portion having a second diameter, and a third portion having a third diameter, the first, second and third portions being arranged in an alternating fashion between the proximal end and the distal end of the wire guide along at least a portion of the wire guide.

16. The wire guide device of claim 15 wherein the wire guide device includes a cross-sectional shape selected from the group consisting of circular, round, oval and square.

17. The wire guide device of claim 15 wherein the wire guide device includes a cross-sectional shape selected from the group consisting of triangular, rectangular, pentagonal and hexagonal.

18. The wire guide device of claim 15 wherein the first diameter is smaller than the second diameter.

19. The wire guide device of claim 15 wherein the third diameter is larger than the first diameter and the second diameter.

20. The wire guide device of claim 15, wherein the third portion is configured to engage a lumen so as to reduce the friction along the lumen.

21. A wire guide device comprising a first plurality of diameters, a second plurality of diameters, and a third plurality of diameters, the first, second and third plurality of diameters being arranged in an alternating fashion along a portion of wire guide.
